(19) United States
(12) Patent Application Publication
(10) Pub. No.: US 2023/0183656 A1
(43) Pub. Date: Jun. 15, 2023

(54) METHODS AND COMPOSITIONS FOR PURIFYING ADENO ASSOCIATED VIRUS PARTICLES OR ADENOVIRUSES

(71) Applicant: Merck Patent GmbH, Darmstadt (DE)

(72) Inventors: Moritz LEY, Darmstadt (DE); Eric WEISS, Burlington, MA (US); Ashish SAKSULE, Burlington, MA (US); Dongli GUAN, Burlington, MA (US); Herbert LUTZ, Burlington, MA (US)

(73) Assignee: Merck Patent GmbH, Darmstadt (DE)

(21) Appl. No.: 17/924,710

(22) PCT Filed: May 12, 2021
(86) PCT No.: PCT/EP2021/062605

§ 371 Date: Nov. 11, 2022

Related U.S. Application Data
(60) Provisional application No. 63/024,643, filed on May 14, 2020.

(51) Int. Cl. C12N 7/00; C12N 1/06

(52) U.S. Cl. CPC C12N 7/00; C12N 1/06; C12N 2750/14151

(57) ABSTRACT

The present invention relates to compositions and methods for lysing cells and isolating and/or purifying adeno-associated virus particles or adenovirus particles using a detergent selected from the group of alkyldimethylamine oxides.

The present invention relates to compositions and methods for lysing cells and thus purifying adeno-associated virus particles or adenoviruses using a detergent selected from the group of alkyldimethylamine oxides.

Adeno-associated virus (AAV) as well as adenovirus (AdV) have been characterized and developed as a potent viral vector to deliver genes in vitro in cultured cells and also in vivo. AAV is meanwhile a leading platform for in vivo delivery of gene therapies. AdV is used for gene therapy and as vaccine. AAV is a small, non-enveloped virus containing a single-stranded DNA genome of approximately 4.7 kb, consisting of two inverted terminal repeats (ITRs) that are capable of forming T-shape secondary structure and acting as origins of genome replication, one rep region that encodes four overlapping replication proteins, Rep78, Rep68, Rep52, and Rep40, and one cap region that encodes three structural proteins, VP1, VP2, and VP3, and an assembly activating protein (AAP). Naturally isolated serotypes 1-9 of the AAV viruses share the genomic structure although these serotypes may display different tissue tropism. As the AAVs seem to be nonpathogenic, show an efficient transduction and a stable expression, they are regarded as being one of the most promising gene delivery vehicles. Adenoviruses (AdV) are medium-sized, nonenveloped viruses with an icosahedral nucleocapsid containing a double stranded DNA genome. Most AdVs used in gene therapy and vaccine development are derived from serotype 2 (Ad2) and 5 (Ad5).

AAV vectors can be produced in various cell lines in adherent or suspension cell culture formats using transient transfection or co-infection methods. Depending on specific serotypes and production times, viral particles including full, partial and empty species can be secreted out of cells into culture medium or contained inside cells at various ratios.

Initially, stable AAV producer cells were generated by transfection and selection of human-derived cells, like HeLa or HEK293 cells, with an rAAV transfer vector containing the ITR cassette and a packaging construct containing Rep and Cap. Production of recombinant AAV vectors (rAAV) was then achieved by infection with auxiliary viruses such as adenoviruses (AdV) that provide the helper function. After identification of AdV genes required for AAV vector packaging, a helper virus-free method was established using a duo or triple transfection protocol consisting of two or three plasmids including a constructed helper plasmid instead of an auxiliary virus. This system is widely used in research and drug development. In addition, development of baculovirus expression vectors provides another method to produce rAAV viruses in insect Sf9 cells. These different technologies are shown to be able to produce sufficient quantities of rAAV viruses for use in laboratories and clinical trials.

AdV vectors can also be produced in adherent or suspension cell culture formats by HEK293 or PER.C6 cells or derivatives. Due to a large genome of 26 - 45 kb, re-construction of essential viral genes into a few plasmids was not feasible, and production of recombinant AdV has been realized through a single infection of the same viral vector.

A cell lysis step is generally required at harvest to release viral particles into the supernatant. For this application, typical cell lysis reagents such as Triton X-100, Tween 20, and NaCl are broadly utilized. However, for certain serotypes (e.g. AAV2), viral particles tend to be tightly associated with insoluble cellular components thus limit the efficiency of certain cell lysis reagents in terms of virus release.

Current methods of releasing viruses, especially cell-associated viruses, are often time consuming like e.g. physical lysis methods are difficult to scale, or can introduce unwanted and difficult to remove impurities, e.g. Triton X-100. A degradation product of Triton X-100 is octylphenol, an ecotoxin with demonstrated estrogenic effects. Therefore it would be favorable to have an effective reagent and method to lyse the cells and isolate and/or purify viruses like AAV and AdV from the cells in which they are produced whereby the method should not necessitate the use of a reagent that is ecologically questionable.

The inventors have surprisingly found that a certain group of ecofriendly detergents is especially suitable for releasing cell associated viruses. In combination with a salt like sodium chloride they are even more effective than the known procedures with Triton X100.

The present invention is thus directed to a method for lysing cells enclosing adeno-associated virus (AAV) particles or adenovirus particles by contacting a suspension of said cells with an effective amount of a composition comprising an alkyldimethylamine oxide to promote cell lysis and release of the AAV particles from the cells whereby the viral particles remain unaffected.

Typically this lysis method is part of a method for purifying viruses whereby further isolation or purification steps are performed in addition to the lysis step.

The present invention is further directed to a method for purifying viral particles of an adeno-associated virus (AAV) or an adenovirus (AdV) from a sample comprising cells enclosing the viral particles by
a) contacting a suspension of the cells and the viral particles with an effective amount of a composition comprising an alkyldimethylamine oxide to promote cell lysis and release of the AAV or AdV particles from the cells
b) isolating and/or purifying the AAV or AdV particles.

In a preferred embodiment, the suspension is contacted with an effective amount of a composition comprising an alkyldimethylamine oxide and a salt like sodium chloride.

In a very preferred embodiment the composition comprises LDAO (Lauryldimethylamine-N-Oxide) and/or TDAO (Tetradecyl Dimethylamine-N-Oxide) and sodium chloride and/or potassium chloride. Especially preferred is a composition comprising LDAO (Lauryldimethylamine-N-Oxide) and/or TDAO (Tetradecyl Dimethylamine-N-Oxide) and sodium chloride.

In another preferred embodiment, in step a) the cells are contacted with the composition for 30 to 180 minutes, preferably for 60 to 90 minutes.

In another embodiment the viral particles are AAVs, especially an AAV2 or AAV9 serotype.

In one embodiment the composition is an aqueous solution comprising alkyldimethylamine oxide and a salt like sodium chloride. Preferably, the composition contains only water, one or more alkyldimethylamine oxides, sodium chloride and optionally buffer components.

In a preferred embodiment, the concentration of the alkyldimethylamine oxide in the composition is such that the concentration in the mixture with the cells is between 1% and 4% (w/w).

In another preferred embodiment the concentration of the salt like sodium chloride in the composition is such that the concentration in the mixture with the cells is between 0.05 and 1 mol/l.

In one embodiment step b) is performed by filtration or centrifugation.

In one embodiment, the method of the present invention comprises one or more of the following steps:
Clarification
filtration
dialysis/ diafiltration
tangential flow filtration
treatment with nuclease, e.g. RNase and/or DNase
treatment with chloroform
ion exchange chromatography
affinity chromatography
hydrophobic interaction chromatography
centrifugation
PEG precipitation

The present invention is further directed to a composition comprising one or more alkyldimethylamine oxides and a salt like sodium chloride.

In a preferred embodiment the composition is an aqueous solution comprising 5 to 30% (w/w) TDAO and/or LDAO and 1 to 6 mol/l NaCl.

In a preferred embodiment the composition has a pH between 6 and 9.

The present invention is further directed to a kit comprising the composition of the present invention as well as a nuclease.

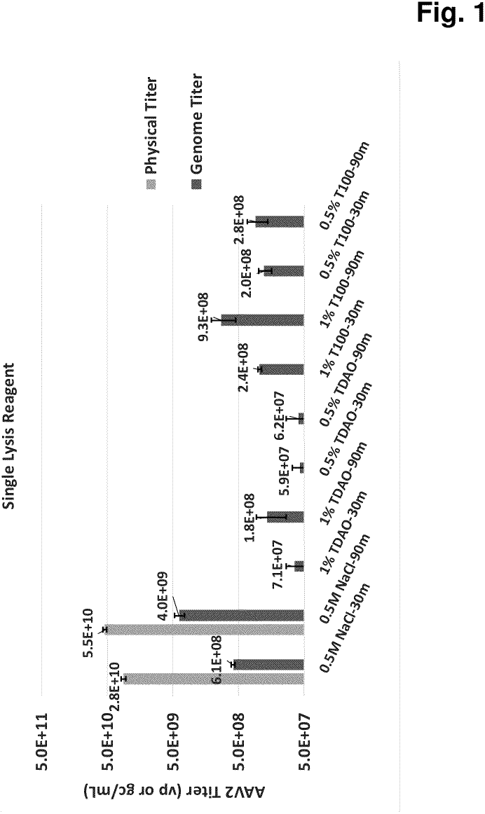

FIG. 1 shows AAV2 titers after cell lysis with single reagents. Further details can be found in Example 1.

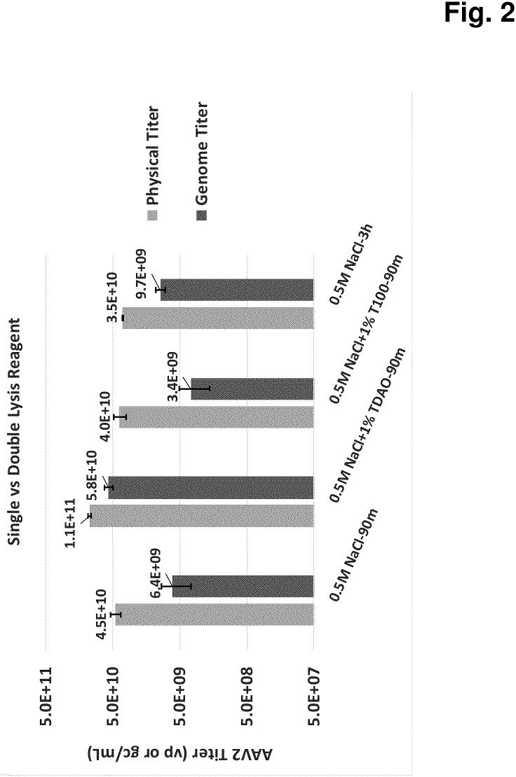

FIG. 2 shows AAV2 titers after cell lysis with single and double reagents. Further details can be found in Example 2.

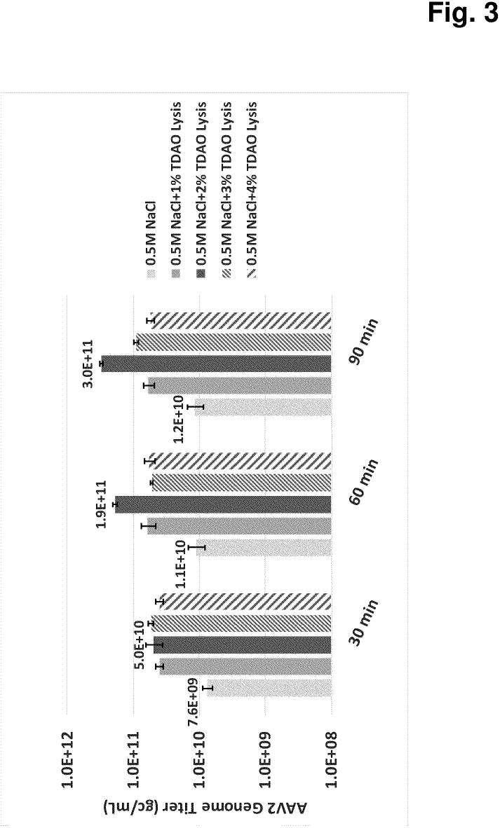

FIG. 3 shows AAV2 genome titers after cell lysis with NaCl and NaCl/TDAO. Further details can be found in Example 4.

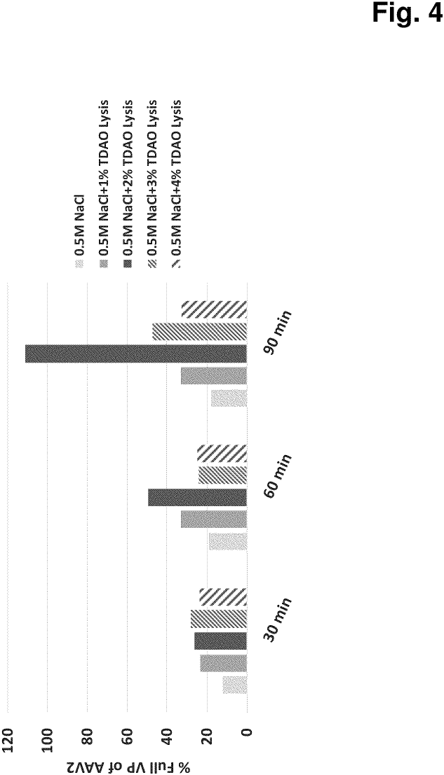

FIG. 4 shows the percentage of full viral particles (vp) as the ratio of genome titer to physical titer. Further details can be found in Example 4.

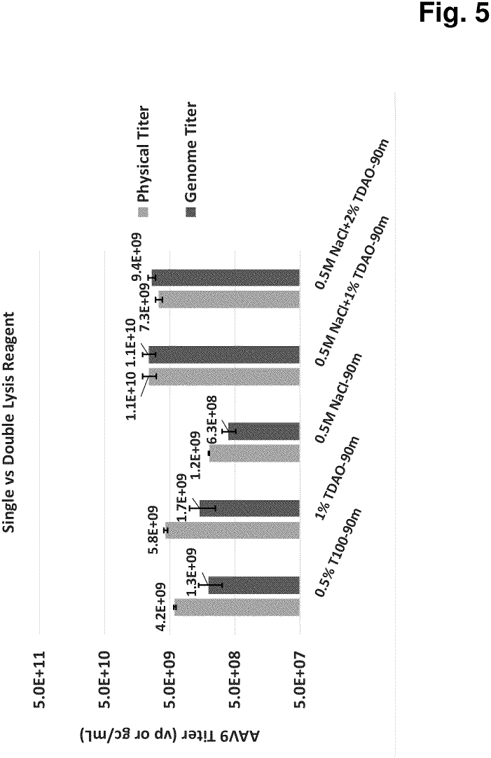

FIG. 5 shows AAV9 titers after cell lysis with single and double reagents. Further details can be found in Example 5.

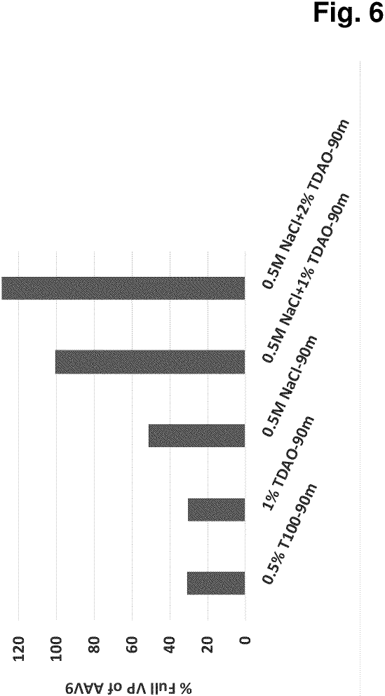

FIG. 6 shows the percentage of full viral particles (vp) as the ratio of genome titer to physical titer. Further details can be found in Example 5.

Alkyldimethylamine oxides suitable for lysing the virus producing cells are amphipathic, charged amine oxides coupled to saturated hydrocarbon chains of varying lengths. Preferably, the length of the saturated hydrocarbon chain is between 8 and 18 carbon atoms. In a preferred embodiment, the alkyldimethylamine oxides are selected from the group consisting of dimethyldecylamineoxide, dimethylundecylamineoxide, dimethyldodecylamineoxide (LDAO), dimethyltridecylamineoxide and dimethytetradecylamineoxide (TDAO).

Table 1 shows the properties of selected alkyldimethylamine oxides.

TABLE 1

| chemical name | alkyl chain length | CAS | MW g/mol | CMC* mM | CMC wt% | biodegra dation in 28 days |
|---|---|---|---|---|---|---|
| N,N-Dimethyloctyl amine N-oxide | C8 | 2605-78-9 | 173.3 | 150 | 2.60 | 100% |
| N,N-Dimethyldecyl amine N-oxide | C10 | 2605-79-0 | 201.35 | 15 | 0.30 | 97% |
| N,N-Dimethyldode cylamine N-oxide Synonym: Lauryldimethyl amineoxide (LDAO) | C12 | 1643-20-5 | 229.40 | 1.7 | 0.039 | 95% |
| N,N-Dimethyltertra decylamine N-oxide (TDAO) | C14 | 3332-27-2 | 257.46 | 0.268 | 0.0069 | 88% |
| N,N-Dimethylhexa decylamine N-oxide | C16 | 7128-91-8 | 285.52 | 0.025 | 0.00071 | <88% |
| N,N-Dimethylocta decylamine N-oxide | C18 | 2571-88-2 | 313.56 | | | |

These compounds are preferably used at concentrations above their critical micelle concentration. The critical micelle concentration (CMC) is defined as the concentration of detergents above which micelles form and all additional detergents added to the system go to micelles. The value of the CMC for a given detergent in a given medium depends on temperature, pressure, and (sometimes strongly) on the presence and concentration of other surface active substances and electrolytes.

Examples of salts like sodium chloride are salts comprising a metal cation like K+, Na+, Li+, Mg²⁺, Ca²⁺ and an anionic component like F—, SO₄^{2—}, HPO₄^{2—}, acetate, Cl^{—} . Preferred are salts comprising monoatomic ions. Especially preferred are chloride salts like sodium chloride and potassium chloride, whereby sodium chloride is most preferred.

Adeno-associated virus (AAV) is a member of the Parvoviridae family. The AAV genome is composed of a linear single-stranded DNA molecule which contains approximately 4.7 kilobases (kb) and consists of two major open reading frames encoding the non-structural Rep (replication) and structural Cap (capsid) proteins. Flanking the AAV coding regions are two cis-acting inverted terminal repeat (ITR) sequences, approximately 145 nucleotides in length, with interrupted palindromic sequences that can fold into hairpin structures that function as primers during initiation of DNA replication. In addition to their role in DNA replication, the ITR sequences have been shown to be necessary for viral integration, rescue from the host genome, and encapsidation of viral nucleic acid into mature virions (Muzyczka, (1992) Curr. Top. Micro. Immunol. 158:97-129).

Multiple serotypes of AAV exist and offer varied tissue tropism. Known serotypes include, for example, AAV1, AAV2, AAV3, AAV4, AAV5, AAV6, AAV7, AAV8, AAV9, AAV10 and AAV11.

Vectors derived from AAV are particularly attractive for delivering genetic material because they are able to infect (transduce) a wide variety of non-dividing and dividing cell types including muscle fibers and neurons and they are devoid of the virus structural genes, thereby eliminating the natural host cell responses to virus infection, e.g., interferon-mediated responses. In addition, wild-type viruses have never been associated with any pathology in humans.

According to the present invention scAAV are also within the group of AAVs. Self-complementary adeno-associated vectors (scAAV) are viral vectors engineered from the naturally occurring adeno-associated virus (AAV) for use in gene therapy. ScAAV is termed “self-complementary” because the coding region has been designed to form an intramolecular double-stranded DNA template.

Thus, in some embodiments, by an “AAV” is meant a vector or virus derived from an adeno-associated virus serotype, including without limitation, AAV-1, AAV-2, AAV-3, AAV-4, AAV -5, AAV- 6, AAV-7, AAV -8, AAV-9, AAV-10 and AAV-1 1. AAV vectors can have one or more of the AAV wild-type genes deleted in whole or part, e.g., the rep and/or cap genes, but retain functional flanking ITR sequences. Functional ITR sequences are necessary for the rescue, replication and packaging of the AAV virion. Thus, an AAV vector is defined herein to include at least those sequences that provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. In one embodiment, the vector is an AAV-9 vector, with AAV-2 derived ITRs. Also by an “AAV” is meant the protein shell or capsid, which provides an efficient vehicle for delivery of vector nucleic acid to the nucleus of target cells.

The term “AAV” as used herein is intended to also encompass recombinant AAV.

Adenoviruses (AdV) are medium-sized (90-100 nm), nonenveloped (without an outer lipid bilayer) viruses with an icosahedral nucleocapsid containing a double stranded DNA genome. They have a broad range of vertebrate hosts; in humans, more than 50 distinct adenoviral serotypes, belonging to seven species (A-G), have been found to cause a wide range of illnesses. Methods of preparing recombinant AdVs and packaging them into suitable host cells are known in the art. They are used for gene therapy and as vaccines to express foreign antigens. Adenovirus vectors can be replication-defective; certain essential viral genes are deleted and replaced by a cassette that expresses a foreign therapeutic gene. Replication-competent (oncolytic) vectors are employed for cancer gene therapy. Oncolytic vectors are engineered to replicate preferentially in cancer cells and to destroy cancer cells through the natural process of lytic virus replication. Many clinical trials indicate that replication-defective and replication-competent adenovirus vectors are safe and have therapeutic activity.

Adenovirus (AdV) and adeno-associated virus (AAV) are herein also called viruses, viral particles or viral vectors.

As used herein, the term “cell” or “cell line” refers to a single cell or to a population of cells capable of continuous or prolonged growth and division in vitro. In some embodiments, e.g. the terms “HEK293 cells”, “293 cells” or their grammatical equivalents are used interchangeably here and refer to the host/packing cell line used in the methods disclosed herein.

Suitable cells and cell lines have been described for use in production of AAVs and AdVs. The cells themselves may be selected from any biological organism, including prokaryotic (e.g., bacterial) cells and eukaryotic cells including insect cells, yeast cells and mammalian cells. Particularly desirable host cells are selected from among any mammalian species, including, without limitation, A549, WEHI, 3T3, 10T1/2, BHK, MDCK, COS 1, COS 7, BSC 1, BSC 40, BMT 10, VERO, Wl38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster.

Generally, the expression cassette is composed of, at a minimum, a 5′ AAV inverted terminal repeat (ITR), a nucleic acid sequence encoding a desirable therapeutic, immunogen, or antigen operably linked to regulatory sequences which direct expression thereof, and a 3′ AAV ITR. In one embodiment, the 5′ and/or 3′ ITRs of AAV serotype 2 are used. However, 5′ and 3′ ITRs from other suitable sources may be selected. It is this expression cassette that is packaged into capsid proteins to form an AAV virus or particle.

In addition to the expression cassette, the cells contain the sequences which drive expression of AAVs in the cells (cap sequences) and rep sequences of the same source as the source of the AAV ITRs found in the expression cassette, or a cross-complementing source. The AAV cap and rep sequences may be independently selected from different AAV parental sequences and be introduced into the host cell in a suitable manner known to one in the art. While the full-length rep gene may be utilized, it has been found that smaller fragments thereof, i.e., the rep78/68 and the rep52/40 are sufficient to permit replication and packaging of the AAV.

The cells also require helper functions in order to package the AAV of the invention. Optionally, these helper functions may be supplied by a herpesvirus. In another embodiment, the necessary helper functions are each provided from a human or non-human primate adenovirus source, such as are available from a variety of sources, including the American Type Culture Collection (ATCC), Manassas, Va. (US).

A buffer or a buffer solution is a solution of a certain pH used to prevent pH changes. Examples of buffers are CO₂/HCO₃ (carbonate), phosphate, HEPES, PIPES, ACES, BES, TES, MOPS and TRIS.

During the manufacturing of AAVs, a percentage of capsids might not incorporate any of the transgenes and are referred to as empty capsids or empty AAVs. Additionally, capsids that contain fragments of the transgene are called partial capsids or partial AAVs. These undesired product-related impurities are co-produced with the full capsids or full AAVs which contain the full length of the desired transgene. The same wording can be used for AdVs.

Purification means to increase the degree of purity of a target molecule, in this case the AAVs or AdVs, e.g. by removing or reducing the amount of one or more impurities.

The term “impurity” or “contaminant” as used herein, refers to any foreign or objectionable molecules or species, including a biological macromolecules such as DNA, RNA, one or more host cell proteins, nucleic acids, endotoxins, lipids, impurities of synthetic origin like detergents, partial and/or empty AAVs or AdVs as well as one or more additives which may be present in a sample containing the viral particles to be purified and thus to be separated from one or more of the impurities.

A bioreactor is any vessel or tank in which cells can be cultured. Incubation is typically done under suitable conditions like suitable temperature etc and with a suitable medium for supporting the growth/culturing of the cells. A person skilled in the art is aware of suitable incubation conditions for supporting or maintaining the growth/culturing of cells.

The present invention is based on the finding that a certain type of composition comprising at least an alkyldimethylamine oxide based detergent is especially suitable for lysing cells and setting free viral particles. AAV or AdV vectors can be produced in various cell lines in adherent or suspension cell culture formats using transient transfection, infection or co-infection methods. Depending on specific serotypes and production times, viral particles including full, partial and empty species can be secreted out of cells into culture medium or contained inside cells at various ratios. A cell lysis step is generally required at harvest to release viral particles into the supernatant. Sometimes, the viral particles tend to be tightly associated with insoluble cellular components which limits the efficiency of certain cell lysis reagents in terms of virus release.

It has been found that alkyldimethylamine oxides alone or in combination with salts like sodium chloride are especially suitable to promote cell lysis and isolation and/or purification of the viral particles.

The production of cells comprising AAVs or AdVs is known to a person skilled in the art. Typically, the selected cells are expanded in suitable culture media in a bioreactor under suitable conditions. The cells may be grown as adherent or suspension culture. For example, in suspension culture of HEK293 cells suitable seeding numbers before transfection are 0.5 to 1.1 e6 viable cells per ml.

Suitable methods for the transduction are known in the art. In one embodiment, cells can be transduced in vitro by combining a rAAV or AdV with the cells, e.g., in appropriate media, and screening for those cells harboring the DNA of interest using conventional techniques such as Southern blots and/or PCR, or by using selectable markers.

Transfection can be performed using any of the techniques known in the art, including but not limited to electroporation, lipofection, e.g. with a lipofectamine, cationic polymers and cationic lipids. Any suitable transfection media may be used. In one embodiment of the transfection process, adherent or suspension human embryonic kidney (HEK293) cells are transfected with a triple DNA plasmid polyethylenimine (PEI) coprecipitation.

In an embodiment, the present disclosure provides a method for manufacturing an AAV or AdV based viral vector comprising the steps of (i) culturing cells in a bioreactor, (ii) transfecting the cells with plasmids to enable production of the AAV particles, or infecting the cells with an AdV vector to amplify/ produce the same AdV particles, (iii) contacting a mixture of the cells and the viral particles with an effective amount of a composition comprising an alkyldimethylamine oxide and optionally a salt like sodium chloride to promote cell lysis and release of the viral particles from the cells iv) isolating and/or purifying the viral particles.

After a suitable virus production period post transfection or infection, the cells are lysed and the viral particles harvested. In some embodiments, the cells are dissociated from the bioreactor before the cell lysis process is initiated. In some embodiments, the cells are lysed in situ.

According to the present invention, for lysis, the cells are contacted with a composition comprising an alkyldimethylamine oxide and optionally a salt like sodium chloride. Preferably, the lysis solution is added to the bioreactor comprising the suspension of the cells so that a mixture of the cell suspension and the composition is generated.

Incubation of the mixture comprising the cells and the composition comprising an alkyldimethylamine oxide and optionally a salt like sodium chloride is done typically for 30 to 180 minutes, preferably for an incubation time between 60 and 90 minutes. Shorter and longer times may also be appropriate.

The pH of the mixture during the incubation can vary in broad range. It can for example be between pH 4 and pH 10, typically it is between pH 6 and pH 9.

The temperature of the mixture during incubation can also vary in a broad range. It can for example be between 20 and 37° C.

To have an effective amount of a composition comprising an alkyldimethylamine oxide to promote cell lysis and release of the AAV particles from the cells the concentration of the alkyldimethylamine oxide, the composition is such that it effectively induces cell lysis, that means that after the incubation with the composition under suitable conditions as described above, at least 80%, preferably 100% of the cells are lysed. For this, the concentration of the alkyldimethylamine oxide in the final mixture with the cells is preferably above its CMC. For TDAO, N,N-Dimethyltridecylamine N-oxide, the concentration in the mixture with the cells is typically between 0.1% and 5% (w/w), preferably between 1% (w/w) and 4% (w/w). Typically the volume of the composition that is added is smaller than the volume of the cell culture.

Consequently, suitable concentrations of the alkyldimethylamine oxide, especially TDAO, in the composition are between 10 and 30% (w/w). Suitable concentrations of a salt like sodium chloride in the composition to be added to the cell suspension are between 1 and 6 mol/l, typically around 3 to 5 mol/l, so that the final concentration in the mixture with the cell suspension is between 0.05 and 1 mol/l.

In a preferred embodiment, the detergent used is TDAO, N,N-Dimethyltridecylamine N-oxide and/or LDAO, either as single component or as a mixture with NaCl.

In a very preferred embodiment, the composition is an aqueous solution. It might also comprise one or more buffers.

It has been found that the composition as defined above effectively induces lysis of the cells and that especially viral particles which tend to stick to the cells and the cellular debris can be effectively separated from the cells.

After incubation with the lysis composition the released AAVs or AdVs can then be isolated and/or purified. This can be done with any methods. Typically, it is done by one or more method steps including filtration and/or centrifugation.

In one embodiment the mixture is filtered through a filter that removes large molecule contaminants and cellular debris but that permits AAVs to pass therethrough.

In one embodiment, the released viral particles can be separated and purified from the cell culture medium using clarification. Clarification can be a microfiltration process in which relatively larger components such as lysed cells and/or impurities are removed from a solution. Clarification filters include depth filtration, charged depth filtration and similar microfiltration techniques.

Tangential flow filtration can be used to concentrate the mixture of purified viral particles and to remove salts and proteins. Tangential flow filtration (TFF) refers to a generally rapid and efficient method for filtration or purification of a solution containing target product and/or impurities during which a solution or liquid stream flows parallel to a filtering membrane.

Centrifugation can for example be a low speed centrifugation to remove larger particles like cellular debris. This can be for example done at 10000 to 12000 g for 10 to 30 minutes. The released viral particles can be found in the supernatant.

The isolation and/or purification of the AAVs typically includes one or more of the following process steps:
Clarification
filtration
dialysis/ diafiltration
tangential flow filtration
treatment with nuclease, e.g. RNase and/or DNase
treatment with chloroform
ion exchange chromatography
affinity chromatography
hydrophobic interaction chromatography
centrifugation
PEG precipitation

In some embodiments, a nuclease, typically an endonuclease, is added, e.g. to reduce the amount of host cell DNA. It can be added directly to the mixture in the bioreactor before, while or after lysis. The nuclease may be one that degrades both DNA and RNA. In one embodiment, the endonuclease is a genetically engineered endonuclease from Serratia marcescens that is sold under the name Benzonase® (EMD Millipore).

In various embodiments, ion exchange chromatography is applied for further purification. This can for example be an anion (AEX) or a cation exchange (CEX) capture chromatography step. Such a step is used, e.g., to separate the viral particles from host cell proteins, host cell DNA, host cell lipids, detergents and other process-related impurities. The principles of cation and anion exchange chromatography are well known in the art. The sample is loaded and the column is washed with a loading buffer. Finally, an elution buffer is used to elute the sample of interest off the column, and fractions containing the sample are collected.

Other suitable chromatographic methods are hydrophobic interaction chromatography, size exclusion chromatography or affinity chromatography. Instead of bind-elute mode, also flow-through mode can be suitable.

Precipitation of the AAVs or AdVs with polyethylene glycol (PEG) is also possible. For this PEG is added to a viral sample. The molecular weight of the PEG is typically from about 3.000 to about 15.000 g/mol. The concentration of the PEG in the precipitation solution can be adjusted as needed, it can for example be around 5% (w/w).

Chloroform can be added to dissolve lipids and inactivate abundant proteins. The virus particles are not affected by the chloroform which can later be separated by phase separation and/or centrifugation.

Salts like sodium chloride can be removed from the virus particles by dialysis. The virus containing solution can be dialyzed against water or another solution depending on the impurity that shall be removed.

Typical AAV purification processes include clarification, concentration and diafiltration using tangential flow filtration, chromatography purification by using affinity chromatography and ion exchange chromatography. In some processes, ultracentrifugation and gradient ultracentrifugation are used instead of chromatography or in addition to chromatography. Final steps in AAV purification typically involves concentration and diafiltration into suitable excipient buffer composition and sterile filtration.

The present invention is further directed to a composition to be used in the method of the invention comprising an aqueous solution of an alkyldimethylamine oxide and sodium chloride. The concentration of the alkyldimethylamine oxide in the composition is typically above 1% (w/w), preferably between 10% (w/w) and 30% (w/w) so that when the composition is added to the suspension comprising the cells to be lysed, the final concentration of the alkyldimethylamine oxide can be adjusted to being above 0.1% (w/w), preferably between 1 and 4% (w/w).

In a preferred embodiment, the detergent used is TDAO, N,N-Dimethyltridecylamine N-oxide and/or LDAO, either as single component or as a mixture with NaCl. Most preferred is a mixture of TDAO with NaCl.

In a very preferred embodiment, the composition is an aqueous solution. It might also comprise one or more buffers.

The concentration of the salt like sodium chloride in the composition is typically between 1 and 6 mol/l, preferably around 3 to 5 mol/l, so that the final concentration in the mixture with the cell suspension is between 0.05 and 1 mol/l.

In a preferred embodiment, the composition is only made of water, one or more alkyldimethylamine oxides, sodium chloride and/or potassium chloride and optionally a buffer.

The present invention is also directed to a kit comprising the composition of the invention as described above and a nuclease, preferably an endonuclease, most preferred benzonase®. The kit typically comprises two containers, e.g. bottles, with the two components but it may also comprise further components and thus further containers.

As can be seen from the Examples, the method of the present invention is very efficient. Using the method of the invention at least 80%, most preferred 100% of the cells can be lysed while the virus is released. Cell lysis can for example be detected via microscope imaging. The number of virus particles released with the lysis method of the present invention exceeds the number of virus particles released by using Triton X100 or sodium chloride or even a combination thereof. This could not be expected. The method and the composition of the present invention are especially suitable for AAV serotypes that are tightly cell associated.

For example, in an AAV2 suspension upstream process including a cell lysis step using either 0.5% Triton X-100 (T100) or 0.5 M NaCl for 90 min, both at 37° C., significantly reduced viral titers were observed (FIG. 1). In contrast to that, much higher virus titers could be reached when using a lysis solution comprising 0.5 M sodium chloride and 1% TDAO for the same amount of time (see FIG. 2).

Another advantage of the present invention is that the detergent and optionally a salt like sodium chloride that is used for cell lysis and viral release can be removed using tangential flow filtration. In tangential flow filtration, ultrafiltration membranes can be used to purify and concentrate viral particles and perform diafiltration to remove detergent.

The present invention is further illustrated by the following figures and examples, however, without being restricted thereto.

The entire disclosure of all applications, patents, and publications cited above and below as well as U.S. Provisional Pat. Application 63/024,643, filed on May 14, 2020, are hereby incorporated by reference.

### EXAMPLES

The following examples represent practical applications of the invention.

### Example 1 - Comparative Example

An AAV2 suspension upstream process is performed including a cell lysis step using various single reagents, like Triton X-100 (T100) or TDAO at various concentrations or 0.5 M NaCl, all at 37° C. The samples were centrifuged at ~13000 g for 5 min at different time points post lysis, and supernatants were removed and stored at -70° C. for assays. Physical titers were measured by enzyme-linked immunosorbent assay (ELISA) using an AAV2 Titration ELISA kit (Progen) following manufacturer’s protocol, genome titers were measured by quantitative PCR (qPCR) targeting a specific sequence within the transfer vector. All reagents and assay protocols are from MilliporeSigma unless otherwise specified. Among all the conditions tested, 0.5 M NaCl was the only one capable of releasing significant amounts of AAV2 at physical titer of e10 vp/mL and genome titer of e9 gc/mL levels (FIG. 1). Moreover, time-dependent effects from 30 min to 90 min post lysis for all 3 single reagents, and concentration-dependent effects from 0.5% to 1% for both single detergents have been observed (FIG. 1). But none of the single reagents showed satisfying AAV release level typically at physical titer of e11 vp/mL and genome titer of e10 gc/mL.

In FIG. 1, each bar represents an average of 2 dilutions x 2 assay wells or 3 assay wells and error bars are standard deviations.

### Example 2

The same process is performed as in Example 1 but with NaCl combined with different detergents.

It was demonstrated that addition of 1% TDAO but not 1% Triton X-100 to 0.5 M NaCl promoted 3-9 fold increase of AAV2 dissociation (FIG. 2). In addition, a longer lysis time of 3 hr was evaluated to be equivalent compared to the shorter time of 90 min (FIG. 2).

In FIG. 2 each bar represents an average of 2 dilutions x 2 assay wells or 3 assay wells and error bars are standard deviations.

### Example 3

Cell lysis performed according to the general procedure of Example 1 was performed with different reagents and compared in terms of turbidity reduction and lysis efficiency. Notably, combination of 1% TDAO and 0.5 M NaCl generated faster and more complete cell lysis compared to single reagent - 0.5 M NaCl or 0.5% Triton X-100 only. (Table 2).

TABLE 2

| 3L Mobius® at 72 hr PT | | Turbitiy post lysis (NTU) | % cell lysis after 30 min |
|---|---|---|---|
| AAV8 | HEK293-derived cells, 0.5% T100, 30 min | 180.5 | |
| | HEK293T-derived cells, 0.5% T100, 30 min | 312 | |
| AAV2 | HEK293-derived cells, 0.5 M NaCl+1% TDAO, 90 min | 39.5 | |
| | HEK293T-derived cells, 0.5 M NaCl | | 80% |
| | HEK293T-derived cells, 0.5 M NaCl+1% TDAO, 90 min | 99.6 | 100% |

Each turbidity in Table 2 is an average of 1-3 bioreactors x 2 measurements. PT = post transfection. Before lysis, turbidity was ~300-400 NTU for HEK293-derived cells and ~500-600 NTU for HEK293T-derived cells. Cell counts were taken by ViCell XR before lysis and at 30 min post lysis, and % cell lysis was calculated as a ratio of lysed cells to total cells before lysis.

### Example 4

To investigate the optimal concentration of TDAO in combination with 0.5 M NaCl, an AAV2 dissociation test using 0 to 4% of TDAO and including an additional lysis time of 60 min was performed. As shown in FIG. 3, at least 3-5 fold increase of AAV2 dissociation by addition of TDAO to 0.5 M NaCl was confirmed as before, and an optimal concentration of 2% TDAO was identified in combination with 0.5 M NaCl. Furthermore, the best lysis time of 90 min was confirmed as before based on genome titers. When calculating % full viral particles (vp) as the ratio of genome titer to physical titer, it was noted that 2% TDAO also facilitated more release of full viral particles over time with ~100% full VP at 90 min post lysis due to unknown mechanisms (FIG. 4).

In FIG. 3 each bar represents an average of 3 assay wells and error bars are standard deviations. In FIG. 4, each bar represents only one data from one experiment.

### Example 5

An AAV9 suspension upstream process is performed as in Example 1 but using both single reagents and double reagents combined with NaCl.

In contrast to AAV2 results in FIG. 1, either single detergent TDAO or Triton X-100 enhanced AAV9 dissociation 2-5 fold compared to 0.5 M NaCl only (FIG. 5). However, similar to AAV2 results in FIG. 2, combination of 1% TDAO and 0.5 M NaCl promoted 2-17 fold increase of AAV9 dissociation compared with respective single reagent, though no benefits were observed for double reagents with 2% TDAO compared to 1% TDAO (FIG. 5). These results demonstrated although AAV9 and AAV2 might interact with different cellular components in different ways, combination of

TDAO and NaCl could promote AAV dissociation of these different serotypes. It is also consistent between AAV2 and AAV9 that combination of TDAO and NaCl facilitated more release of full viral particles with much higher % full VP than those from single reagent (FIGS. 4 and 6). Nevertheless, an underlying mechanism for such phenomenon is currently unknown.

In FIG. 5, each bar represents an average of 2 dilutions x 2 assay wells or 3 assay wells and error bars are standard deviations. In FIG. 6, each bar represents only one data from one experiment.

1. A method for lysing cells enclosing adeno-associated virus (AAV) particles or adenovirus (AdV) particles by contacting a suspension of said cells with an effective amount of a composition comprising an alkyldimethylamine oxide to promote cell lysis and release of the AAV or AdV particles from the cells.

2. A method for purifying viral particles of adeno-associated virus (AAV) or adenovirus (AdV) from a sample comprising cells enclosing the viral particles by
a) contacting a suspension of the cells and the viral particles with an effective amount of a composition comprising an alkyldimethylamine oxide to promote cell lysis and release of the viral particles from the cells
b) isolating and/or purifying the viral particles.

3. Method according to claim 2 characterized in that the composition comprises LDAO (Lauryldimethylamine-N-Oxide) and/or TDAO (Tetradecyl Dimethylamine-N-Oxide) and sodium chloride.

4. Method according to claim 2, characterized in that in step a) the cells are contacted with the composition for 30 to 180 minutes.

5. Method according to 4 claim 2, characterized in that the viral particles are recombinant adeno associated virus (AAV) particles.

6. Method according to claim 2, characterized in that the concentration of alkyldimethylamine oxide in the mixture of the suspension of the cells and the viral particles with an effective amount of a composition obtained in step a) is between 1 and 4% (w/w).

7. Method according to claim 2 characterized in that the concentration of a salt comprising a metal cation selected from K⁺, Na⁺, Li⁺, Mg²⁺, Ca²⁺ and an anionic component selected from F—, SO₄^{2—}, HPO₄^{2—}, acetate, Cl^{—} in the mixture of the suspension of the cells and the viral particles with an effective amount of a composition obtained in step a) is between 0.05 and 1 mol/l.

8. Method according to claim 2,characterized in that the composition is an aqueous solution comprising an alkyldimethylamine oxide and sodium chloride.

9. Method according to claim 2, characterized in that the concentration of the alkyldimethylamine oxide in the composition is between 10% and 30% (w/w).

10. Method according to claim 2, characterized in that the concentration of sodium chloride in the composition is between 1 and 6 mol/l.

11. Method according to claim 2, characterized in that step b) comprises a filtration and/or centrifugation step.

12. Method according to claim 2, characterized in that the method of the present invention comprises one or more of the following steps:
Clarification and filtration
dialysis and diafiltration
treatment with nuclease, e.g. RNase and/or DNase
treatment with chloroform
ion exchange chromatography
affinity chromatography
hydrophobic interaction chromatography
centrifugation
PEG precipitation.

13. A composition comprising one or more alkyldimethylamine oxides and a salt comprising a metal cation selected from K⁺, Na⁺, Li⁺, Mg²⁺, Ca²⁺ and an anionic component selected from F^{—}, SO₄^{2—}, HPO₄2^{—}, acetate, Cl^{—}.

14. A composition according to claim 13, characterized in that the composition is an aqueous solution comprising 10 to 30% (w/w) TDAO and/or LDAO and 1 to 6 mol/l NaCl.

15. A kit comprising the composition according to claim 13 as well as a nuclease.
